(19) United States
(12) Patent Application Publication
(10) Pub. No.: US 2021/0007611 A1
(43) Pub. Date: Jan. 14, 2021

(54) MANAGEMENT SYSTEM, MANAGEMENT METHOD, AND MANAGEMENT PROGRAM

(71) Applicant: TERUMO KABUSHIKI KAISHA, Tokyo (JP)

(72) Inventors: Shinichiro SUDA, Hadano Kanagawa (JP); Kei HONDA, Hadano Kanagawa (JP)

(21) Appl. No.: 17/037,266

(22) Filed: Sep. 29, 2020

Related U.S. Application Data
(63) Continuation of application No. PCT/JP2019/011783, filed on Mar. 20, 2019.

(30) Foreign Application Priority Data
Mar. 30, 2018 (JP) 2018-068356

(51) Int. Cl. A61B 5/029; A61B 5/00; G16H 40/20; G16H 40/67; G16H 50/30; G16H 50/70; G16H 15/00; G16H 50/50

(52) U.S. Cl. CPC A61B 5/029; A61B 5/743; A61B 5/6852; G16H 40/20; G16H 50/50; G16H 50/30; G16H 50/70; G16H 15/00; G16H 40/67

(57) ABSTRACT

A management system includes an acquisition unit that acquires first measurement data of a first circulatory parameter related to symptoms of heart failure from an ICU device and second measurement data of a second circulatory parameter related to symptoms of heart failure from a general ward device at a plurality of timings, and a conversion unit that converts the second measurement data of the second circulatory parameter measured by the general ward device into an estimated value of the first circulatory parameter, based on correspondence between the first measurement data and the second measurement data measured at substantially the same time.

### CROSS-REFERENCE TO RELATED APPLICATION(S)

This application is a continuation of International Patent Application No. PCT/JP2019/011783, filed on Mar. 20, 2019, which is based upon and claims the benefit of priority from Japanese Patent Application No. 2018-068356, filed on Mar. 30, 2018, the entire contents of which are incorporated herein by reference.

### BACKGROUND

### Technical Field

Embodiments relate to a management system, a management method, and a management program for heart failure patients.

### Related Art

Chronic heart failure refers to a condition in which a pumping function of the heart is degraded due to structural abnormality or functional abnormality in the heart but apparently stable hemodynamics are maintained by the function of a compensatory mechanism centering on the kidney. If the hemodynamic balance maintained by the compensatory mechanism is disrupted for some reason, a rise in ventricular filling pressure, a sudden decline in cardiac function, and pulmonary congestion or pulmonary edema triggered by abnormally high sympathetic nerves occur, or perfusion failure (congestion) occurs in the major organ or the whole body. A condition in which such symptoms surface due to acute exacerbation is called acute heart failure.

There are various stages of treatment for acute heart failure, depending on the severity, the purpose of the treatment, and the like. For example, during the acute/lifesaving treatment stage, lifesaving, relief of major symptoms such as respiratory distress, and the like are performed in an emergency room (ER), an intensive care unit (ICU), and the like. During this stage, in order to monitor for a sudden change in the physical condition of a patient, a pulmonary artery catheter disclosed in JP 2018-506328 A is used to measure circulatory parameters related to the symptoms of heart failure, such as central venous pressure, to manage the patient.

After the acute/lifesaving treatment stage, treatment is performed in a general ward or the like to suppress the clinical symptoms of heart failure and reduce the risk of re-exacerbation. At this stage, in order to monitor whether the patient's condition is becoming better or worse, circulatory parameters related to the symptoms of heart failure may be measured to manage the patient. In a general ward or the like, because there is less monitoring by the medical staff, a device based on a measurement principle or a measurement method different from that of the pulmonary artery catheter disclosed in JP 2018-506328 A, such as echocardiography, may be used.

As described as an example of the treatment in the ER or the ICU and the general ward, during the treatment of heart failure, a plurality of different measurement devices may be used to manage the patient. Therefore, even if the measurement devices measure the same circulatory parameter at the same time, a difference between the measurement values may occur due to a difference between the measurement methods of the measurement devices. For this reason, it is difficult to monitor a series of changes in circulatory parameters.

### SUMMARY

In view of the aforementioned circumstances, embodiments provide a management system, a management method, and a management program for heart failure patients, which are configured to monitor a series of changes in circulatory parameters measured by different measurement devices.

A management system according to an embodiment is a management system for managing a patient with heart failure, and includes an acquisition unit that acquires first measurement data of a first circulatory parameter related to symptoms of heart failure from a first measurement device and second measurement data of a second circulatory parameter related to symptoms of heart failure from a second measurement device at a plurality of timings, and a conversion unit that converts the second measurement data of the second circulatory parameter measured by the second measurement device into an estimated value of the first circulatory parameter, based on correspondence between the first measurement data and the second measurement data measured at substantially the same time.

In addition, a management method according to an embodiment is a management method for a patient with heart failure, and includes acquiring at a plurality of timings first measurement data of a first circulatory parameter related to symptoms of heart failure from a first measurement device and second measurement data of a second circulatory parameter related to symptoms of heart failure from a second measurement device, and converting the second measurement data of the second circulatory parameter measured by the second measurement device into an estimated value of the first circulatory parameter, based on correspondence between the first measurement data and the second measurement data measured at substantially the same time.

In addition, a management program according to an embodiment is a management program for a patient with heart failure, which is executed on a processor to carry out a procedure for acquiring at a plurality of timings first measurement data of a first circulatory parameter related to symptoms of heart failure from a first measurement device and second measurement data of a second circulatory parameter related to symptoms of heart failure from a second measurement device used the treatment stage, and a procedure for converting the second measurement data of the second circulatory parameter measured by the second measurement device into an estimated value of the first circulatory parameter, based on correspondence between the first measurement data and the second measurement data measured at substantially the same time.

According to the embodiments, it is possible to monitor a series of changes in circulatory parameters measured by the first measurement device and the second measurement device.

### BRIEF DESCRIPTION OF DRAWINGS

FIG. 1 is a diagram illustrating an outline of a management system according to a first embodiment;

FIG. 2 is a block diagram illustrating the hardware configuration of a server included in the management system according to the first embodiment;

FIG. 3 is a block diagram illustrating the functional configuration of a CPU of the server included in the management system according to the first embodiment;

FIG. 4 is a diagram showing a graph created by the management system according to the first embodiment;

FIG. 5 is a flowchart illustrating a management method according to the first embodiment;

FIG. 6 is a diagram illustrating an outline of a management system according to a second embodiment;

FIG. 7 is a diagram showing a graph created by the management system according to the second embodiment;

FIG. 8 is a flowchart illustrating a management method according to the second embodiment;

FIG. 9 is a diagram illustrating an outline of a management system according to a third embodiment;

FIG. 10 is a diagram showing a graph created by the management system according to the third embodiment;

FIG. 11A is a flowchart illustrating a management method according to the third embodiment; and

FIG. 11B is a continuation of the flowchart in FIG. 11A.

### DETAILED DESCRIPTION

Hereinafter, embodiments will be described with reference to the accompanying diagrams. In the description of the diagrams, the same elements are denoted by the same reference numerals, and repeated description thereof will be omitted. In addition, the dimensional ratios in the diagrams are exaggerated for convenience of explanation, and may be different from the actual ratios.

### First Embodiment

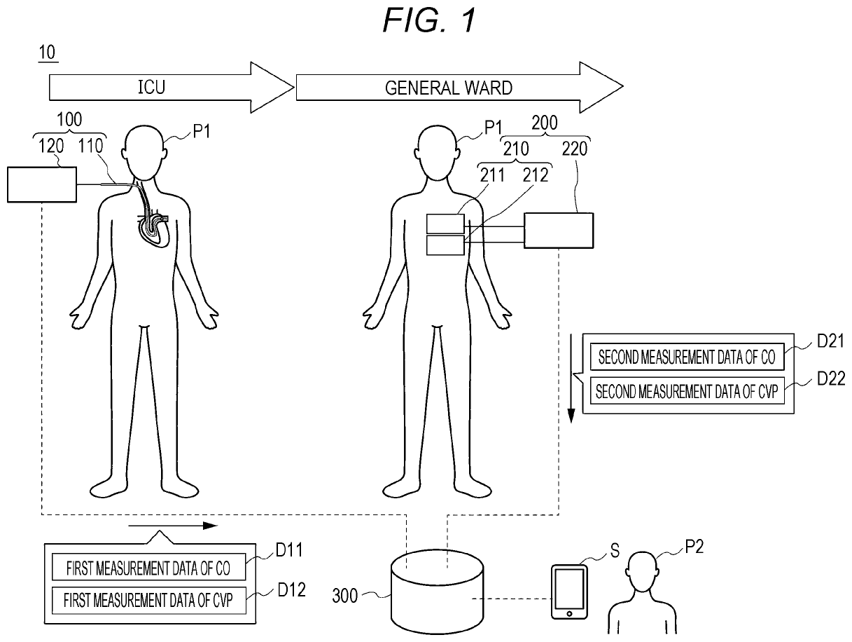

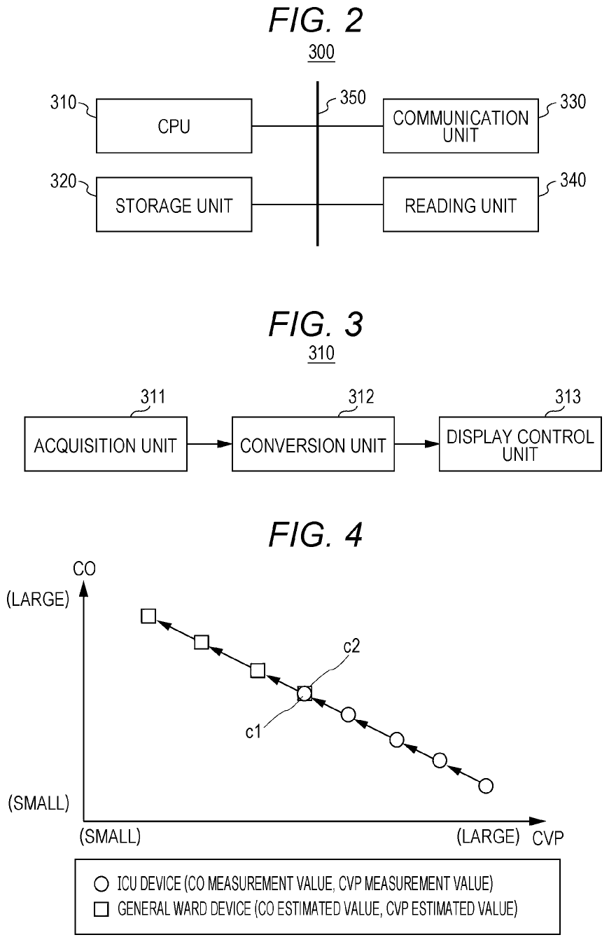

FIG. 1 is a diagram describing the overall configuration of a management system 10 according to a first embodiment. FIGS. 2 and 3 are diagrams describing each unit of the management system 10 according to the first embodiment. FIG. 4 is a diagram describing a graph created by the management system 10.

In the case of acute exacerbation, a patient is first carried to the ICU in many cases. During the treatment stage in the ICU, lifesaving, relief of major symptoms such as respiratory distress, administration of cardiotonic agents, vasodilators, and diuretics, positive pressure ventilation, and the like are performed. In addition, hemofiltration, such as continuous hemodiafiltration (CHDF) or extracorporeal ultrafiltration (ECUM), may be performed when necessary.

After overcoming the acute/lifesaving stage, the patient is moved to the general ward. During the treatment stage in the general ward, treatment is performed to reduce the clinical symptoms of heart failure and reduce the risk of re-exacerbation. In particular, it is known that the prognosis after discharge is poor if the patient is discharged from the hospital while body fluid is accumulated in the body. Therefore, treatment to remove water using a diuretic is performed while checking for excess water content. When the cardiac function is stable, cardiac rehabilitation is further performed at the bedside or in a rehabilitation room to improve exercise endurance or activities of daily living (ADL). In addition, when the cardiac function is stable, prescription, such as β-blocker or ACE inhibitor for resting the heart, may be given in order to improve the life prognosis.

As illustrated in FIG. 1, the management system 10 according to the first embodiment is configured as a system that acquires measurement data of circulatory parameters related to the symptoms of heart failure of a patient P1 having heart failure during a treatment stage in the ICU and a treatment stage in a general ward and provides a series of changes in the circulatory parameters to the user of the management system 10, such as a doctor P2.

The management system 10 will be briefly described with reference to FIG. 1. The management system 10 includes an ICU device 100 (corresponding to a “first measurement device”) used during a treatment stage in the ICU, a general ward device 200 (corresponding to a “second measurement device”) used during a treatment stage in a general ward, and a server 300 that is connected to the ICU device 100, the general ward device 200, and an operation terminal S of the doctor P2 through a network (shown by the broken line in the diagram) to perform transmission and reception of data between the ICU device 100, the general ward device 200, and the operation terminal S of the doctor P2. Hereinafter, each unit of the management system 10 will be described in detail.

(ICU Device)

In the present embodiment, the ICU device 100 includes a pulmonary artery catheter 110 and a control unit 120 that is electrically connected to the pulmonary artery catheter 110 and that controls the operation of the pulmonary artery catheter 110. Hereinafter, each unit of the ICU device 100 will be described.

The pulmonary artery catheter 110 is percutaneously inserted into the blood vessel of the patient P1 through the femoral vein, brachial vein, subclavian vein, internal jugular vein, and the like, and left in the blood vessel such as the pulmonary artery. The pulmonary artery catheter 110 measures a circulatory parameter indicating the cardiac output or the cardiac function and a circulatory parameter indicating the preload or afterload of the heart as first circulatory parameters related to the symptoms of heart failure. As described above, in the present embodiment, the ICU device 100 is an invasive measurement device. Therefore, the ICU device 100 can measure the circulatory parameters with higher accuracy than non-invasive measurement devices. During the treatment stage in the ICU, it is necessary to obtain detailed information regarding the patient's condition in order to check for the risk of sudden degradation of the patient's condition or to perform active treatment. For this reason, the pulmonary artery catheter 110, which is configured to check the condition more directly, is often used even though the degree of invasiveness is high. In addition, in this specification, the “invasive measurement device” means a device having at least a part that is inserted into the living body. In addition, the “non-invasive measurement device” means a device that is not inserted into the living body.

The circulatory parameter indicating the cardiac output or the cardiac function is not limited to a particular parameter so long as it indicates the cardiac output or the cardiac function. For example, cardiac output (CO), stroke volume (SV), and cardiac index (CI) can be used.

The circulatory parameter indicating the preload or afterload of the heart is not limited to a particular parameter so long as it indicates the preload or afterload of the heart. For example, a parameter of body water, a parameter of open blood pressure, and other parameters can be used. In addition, in this specification, the “preload of the heart” means a load applied to the ventricles immediately before the heart contracts. The preload also includes a circulating blood volume. In addition, the “afterload of the heart” means a load applied to the heart immediately after the heart contracts. The afterload also includes changes in peripheral vascular resistance associated with sympathetic hyperactivity and blood flow retention or pressure load in the heart due to heart valve dysfunction.

Examples of the parameter of body water include extracellular water (ECW), intracellular water (ICW), total body water (TBW) that is the sum of ECW and ICW, and weight.

Examples of the parameter of open blood pressure include central venous pressure (CVP), pulmonary arterial pressure (PAP), pulmonary capillary wedge pressure (PCWP), arterial blood pressure (ART), right arterial pressure (RAP), right ventricular pressure (RVP), left arterial pressure (LAP), and left ventricular pressure (LVP).

Other parameters include central venous pressure at end-expiration and pulse pressure variation (PPV).

In the present embodiment, the pulmonary artery catheter 110 includes a temperature sensor (not illustrated) or the like at its distal end, and measures the CO as a circulatory parameter indicating the cardiac output or the cardiac function using the temperature sensor or the like. The pulmonary artery catheter 110 includes a piezoelectric element (not illustrated) or the like at its distal end, and measures the CVP as a circulatory parameter indicating the preload or afterload of the heart using the piezoelectric element or the like. However, the combination of the circulatory parameter indicating the cardiac output or the cardiac function measured by the ICU device 100 and the circulatory parameter indicating the preload or afterload of the heart is not limited to a particular combination. In addition, the ICU device 100 may measure only one of the circulatory parameter indicating the cardiac output or the cardiac function, or the circulatory parameter indicating the preload or afterload of the heart.

The control unit 120 is configured as a known microcomputer including a central processing unit (CPU), a read only memory (ROM) that stores various programs or various kinds of data, and a random access memory (RAM) that temporarily stores programs or data.

The control unit 120 is electrically connected to the temperature sensor, the piezoelectric element, and the like of the pulmonary artery catheter 110, and controls the measurement operation of the pulmonary artery catheter 110. The control unit 120 causes the pulmonary artery catheter 110 to measure the CO and the CVP while the patient P1 is being treated in the ICU. As illustrated in FIG. 1, the control unit 120 transmits, to the server 300, measurement data of the CO (corresponding to “first measurement data”; hereinafter referred to as “first measurement data D11 of CO”) measured by the pulmonary artery catheter 110 and measurement data of the CVP (corresponding to “first measurement data”; hereinafter referred to as “first measurement data D12 of CVP”) measured by the pulmonary artery catheter 110.

(General Ward Device)

In the present embodiment, the general ward device 200 includes a measurement unit 210 configured to measure the second circulatory parameter related to the symptoms of heart failure and a control unit 220 that is electrically connected to the measurement unit 210 and controls the operation of the measurement unit 210. Hereinafter, each unit of the general ward device 200 will be described.

The measurement unit 210 measures a circulatory parameter indicating the cardiac output or the cardiac function and a circulatory parameter indicating the preload or afterload of the heart as second circulatory parameters related to the symptoms of heart failure.

In the present embodiment, the measurement unit 210 includes a CO measurement unit 211 configured to measure the CO as a circulatory parameter indicating the cardiac output or the cardiac function and a CVP measurement unit 212 configured to measure the CVP as a circulatory parameter indicating the preload or afterload of the heart. The CO measurement unit 211 is not limited to a particular device so long as the CO itself is measured or a parameter by which the CO can be estimated is measured. In the present embodiment, the CO measurement unit 211 is a non-invasive measurement device that estimates the CO from echocardiography, chest impedance, arterial pressure waveform, and the like. Examples of the CO measurement unit 211 are described in U.S. patent application Ser. No. 13/874,686 and U.S. patent application Ser. No. 16/607,056, both of which are incorporated by reference herein. The CVP measurement unit 212 is not limited to a particular device so long as the CVP itself is measured or a parameter by which the CVP can be estimated is measured. In the present embodiment, the CVP measurement unit 212 is a non-invasive measurement device, such as an echocardiography device. In a general ward, because of lack of monitoring system by medical staff compared with the ICU, it is difficult to use an invasive measurement device or a measurement device that needs to be monitored at the time of use. For this reason, a non-invasive device is often used as described above. In addition, the CO measurement unit 211 and the CVP measurement unit 212 do not need to be separate measurement units as illustrated in FIG. 1, and may be one measurement unit. In addition, the CO measurement unit 211 and the CVP measurement unit 212 may be invasive measurement devices. For example, the CVP measurement unit 212 may be configured by a known pressure measurement device such as a tube inserted into the living body and a manometer or a pressure transducer configured to measure the internal pressure of the tube.

As described above, the general ward device 200 measures the CO and the CVP in a measurement method different from that of the ICU device 100. In addition, the combination of the circulatory parameter indicating the cardiac output or the cardiac function measured by the general ward device 200 and the circulatory parameter indicating the preload or afterload of the heart is not limited to a particular combination. In addition, the general ward device 200 may measure only one of the circulatory parameter indicating the cardiac output or the cardiac function or the circulatory parameter indicating the preload or afterload of the heart.

The control unit 220 is configured as a known microcomputer including a CPU, a ROM, a RAM, and the like.

The control unit 220 is electrically connected to the CO measurement unit 211 and the CVP measurement unit 212, and controls the measurement operations of the CO measurement unit 211 and the CVP measurement unit 212. The control unit 220 causes the CO measurement unit 211 and the CVP measurement unit 212 to measure the CO and the CVP while the patient P1 is being treated in a general ward. The control unit 220 transmits, to the server 300, measurement data of the CO (corresponding to “second measurement data”; hereinafter referred to as “second measurement data D21 of CO”) measured by the measurement unit 210 and measurement data of the CVP (corresponding to “second measurement data”; hereinafter referred to as “second measurement data D22 of CVP”) measured by the measurement unit 210.

(Server)

As illustrated in FIG. 2, the server 300 includes a CPU 310, a storage unit 320, a communication unit 330, and a reading unit 340. The CPU 310, the storage unit 320, the communication unit 330, and the reading unit 340 are connected to a bus 350, so that transmission and reception of data and the like can be realized through the bus 350. Hereinafter, each unit of the server 300 will be described.

The CPU 310 performs control of each unit, various kinds of arithmetic processing, and the like according to various programs stored in the storage unit 320.

The storage unit 320 includes a ROM, a RAM, and a hard disk that stores various programs including an operating system and various kinds of data. The storage unit 320 stores various programs, such as a management program for managing the patient P1 having heart failure, and various kinds of data.

The communication unit 330 is an interface circuit for communicating with the ICU device 100, the general ward device 200, the operation terminal S of the doctor P2, and the like.

The reading unit 340 reads a management program and the like recorded on a computer-readable recording medium. The computer-readable recording medium is not particularly limited. For example, the computer-readable recording medium can be an optical disk such as a CD-ROM or a DVD-ROM, a USB memory, or an SD memory card. The reading unit 340 is not limited to a particular device. For example, the reading unit 340 can be a CD-ROM drive or a DVD-ROM drive. In addition, the management program may be provided to the server 300 online through a network, such as the Internet.

Next, the main functions of the CPU 310 will be described.

The CPU 310 executes the management program stored in the storage unit 320 to function as an acquisition unit 311, a conversion unit 312, and a display control unit 313 as illustrated in FIG. 3. Hereinafter, each function of the CPU 310 will be described.

First, the acquisition unit 311 will be described.

As illustrated in FIG. 1, the acquisition unit 311 acquires the first measurement data D11 of the CO and the first measurement data D12 of the CVP from the ICU device 100. The acquisition unit 311 acquires the second measurement data D21 of the CO and the second measurement data D22 of the CVP from the general ward device 200. The acquisition unit 311 stores the acquired measurement data D11, D12, D21, and D22 in the storage unit 320.

Next, the conversion unit 312 will be described.

Based on the correspondence between the first measurement data D11 and the second measurement data D21 of the CO, both measured at the same time, the conversion unit 312 converts the second measurement data D21 of the CO measured by the general ward device 200 into a value of the CO that is estimated based on the assumption that the CO is being measured by the ICU device 100. Based on the correspondence between the first measurement data D12 and the second measurement data D22 of the CVP measured at the same time, the conversion unit 312 converts the second measurement data D22 of the CVP measured by the general ward device 200 into a value of the CVP that is estimated based on the assumption that the CVP is being measured by the ICU device 100. Hereinafter, the conversion method will be described in detail.

In this specification, the “same time” means a period during which the symptoms of heart failure of the patient P1 do not significantly change. Therefore, “measurement at the same time” includes not only simultaneous measurement but also measurement at different timings during a period in which the symptoms of heart failure of the patient P1 do not significantly change. In addition, it is preferable that the conversion is performed based on the correspondence between the first measurement data D11 and D12 and the second measurement data D21 and D22 measured at timings closest to each other. In addition, when there is a difference in measurement accuracy between the pieces of first measurement data D11 and D12 and/or between the pieces of second measurement data D21 and D22, it is preferable that the correspondence is established based on data with high measurement accuracy in both the first measurement data and the second measurement data.

Using the first measurement data D11 and the second measurement data D21 of the CO measured at the same time, the conversion unit 312 calculates Conversion Expression Y1 for converting the second measurement data D21 of the CO into a value of the CO that is estimated based on the assumption that the CO is being measured by the ICU device 100. In the present embodiment, as shown in the following Expression 1, a conversion method will be described by taking a case, in which Conversion Expression Y1 is a linear function (proportional expression) having an intercept of 0, as an example. In addition, Conversion Expression Y1 may be any expression as long as the second measurement data D21 of the CO can be converted into a value of the CO that is estimated based on the assumption that the CO is being measured by the ICU device 100, and is not limited to a linear function.

[Expression 1]

Conversion expression Y1: Estimated value of CO=Conversion ratio of CO×Second measurement data D21 of CO (Formula 1)

As shown in the following Expression 2, the conversion unit 312 calculates the ratio between the second measurement data D21 of the CO measured at the same time and the first measurement data D11 of the CO measured at the same time as the conversion ratio of the CO.

$$\begin{matrix} [ Expression 2 ] & \\ Conversion ratio of CO = \frac{ \begin{matrix}First measurement data D 11 of CO \\ measured at same time\end{matrix}}{\begin{matrix}Second measurement data D 21 of CO \\ measured at same time\end{matrix}} & ( Formula 2 )\end{matrix}$$

As shown in the above Expression 1, the conversion unit 312 sets a value, which is obtained by multiplying the second measurement data D21 of the CO measured at a timing other than the same time by the conversion ratio of the CO, as a value of the CO that is estimated based on the assumption that the CO is being measured by the ICU device 100.

Using the first measurement data D12 and the second measurement data D22 of the CVP measured at the same time, the conversion unit 312 calculates Conversion Expression Y2 for converting the second measurement data D22 of the CVP into a value of the CVP that is estimated based on the assumption that the CVP is being measured by the ICU device 100. In the present embodiment, as shown in the following Expression 3, a conversion method will be described by taking a case, in which Conversion Expression Y2 is a linear function (proportional expression) having an intercept of 0, as an example. In addition, Conversion Expression Y2 may be any expression as long as the second measurement data D22 of the CVP can be converted into a value of the CVP that is estimated based on the assumption that the CVP is being measured by the ICU device 100, and is not limited to a linear function.

[Expression 3]

Conversion expression Y2: Estimated value of CVP=Conversion ratio of CVP×Second measurement data D22 of CVP (Formula 3)

As shown in the following Expression 4, the conversion unit 312 calculates the ratio between the second measurement data D22 of the CVP measured at the same time and the first measurement data D12 of the CVP measured at the same time as the conversion ratio of the CVP.

$$\begin{matrix} [ Expression 4 ] & \\ Conversion ratio of CVP = \frac{\begin{matrix}First measurement data D 12 of \\ CVP measured at same time\end{matrix}}{\begin{matrix}Second measurement data D 22 of CVP \\ measured at same time\end{matrix}} & ( Formula 4 )\end{matrix}$$

As shown in the above Expression 3, the conversion unit 312 sets a value, which is obtained by multiplying the second measurement data D22 of the CVP measured at a timing other than the same time by the conversion ratio of the CVP, as a value of the CVP that is estimated based on the assumption that the CVP is being measured by the ICU device 100.

In addition, although the conversion unit 312 converts the second measurement data D21 and D22 measured by the general ward device 200 into values of the CO and the CVP that are estimated based on the assumption that the CO and the CVP are being measured by the ICU device 100 in the present embodiment, the first measurement data D11 and D12 measured by the ICU device 100 may be converted into values of the CO and the CVP that are estimated based on the assumption that the CO and the CVP are being measured by the general ward device 200. Alternatively, the conversion unit 312 may convert the second measurement data D21 and D22 measured by the general ward device 200 into values of the CO and the CVP that are estimated based on the assumption that the CO and the CVP are being measured by the ICU device 100, and the first measurement data D11 and D12 measured by the ICU device 100 may be converted into values of the CO and the CVP that are estimated based on the assumption that the CO and the CVP are being measured by the general ward device 200.

As the conversion ratios of the CO and the CVP deviate from 1, the measurement value of the general ward device 200 deviates from the measurement value of the ICU device 100 having higher measurement accuracy. That is, the conversion ratio of the CO indicates the measurement accuracy of the general ward device 200 based on the ICU device 100. The CPU 310 may perform classification according to the degree of measurement accuracy of the general ward device 200 based on the conversion ratios of the CO and the CVP and transmit the result to the operation terminal S of the doctor P2 or the like.

Next, the display control unit 313 will be described.

As shown in FIG. 4, the display control unit 313 displays points (indicated by white circles in FIG. 4) corresponding to the measurement values of the CO and the CVP of the ICU device 100 and points (indicated by white quadrangles in FIG. 4) corresponding to the estimated values of the CO and the CVP of the general ward device 200 in a graph in which the vertical axis (hereinafter also referred to as “first axis”) indicates the CO and the horizontal axis (hereinafter also referred to as “second axis”) indicates the CVP. In addition, the horizontal axis may indicate the CO, and the vertical axis may indicate the CVP.

In addition, the graph shown in FIG. 4 is merely an example. For example, in FIG. 4, both a point c1 of the ICU device 100 at the same time and a point c2 of the general ward device 200 at the same time are shown. However, at least one of the points c1 and c2 may be displayed.

In addition, in FIG. 4, the points of the ICU device 100 and the points of the general ward device 200 have different shapes (circle and quadrangle). However, the points of the ICU device 100 and the general ward device 200 may have the same shape. In addition, in FIG. 4, the changes of the points with time are indicated by arrows. However, the arrows indicating the change with time may not be shown in the graph. In addition, although FIG. 4 shows a case where the CVP decreases with time and the CO increases with time, the changes in the CVP and the CO are not limited to the case shown in the diagram.

(Management Method)

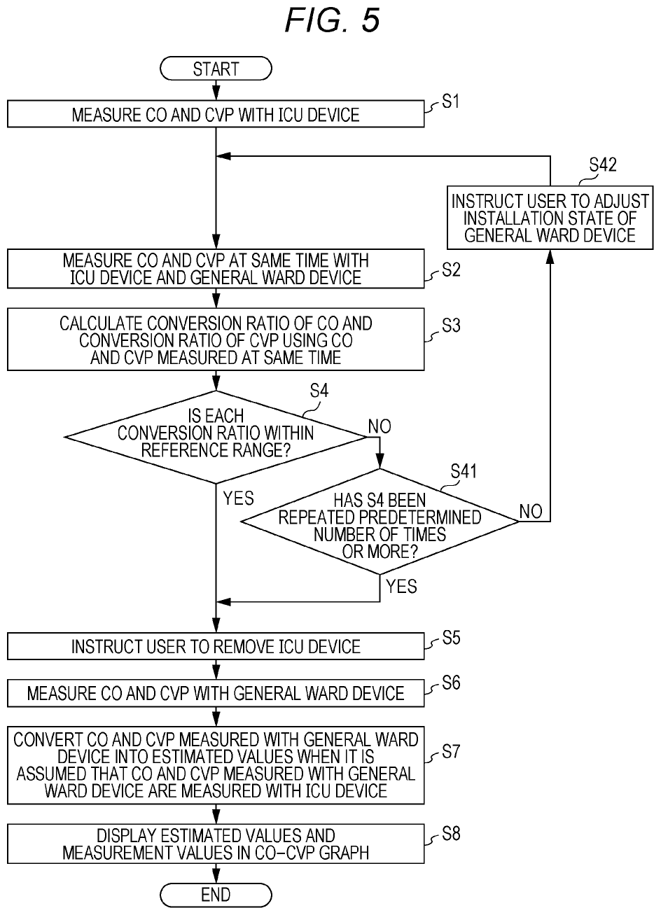

FIG. 5 is a flowchart of the management method according to the present embodiment. Next, the management method according to the present embodiment will be described.

The management method according to the present embodiment will be briefly described with reference to FIG. 5. The management method includes: step S1 of measuring the CO and the CVP during a treatment stage in the ICU; steps S2 to S5 of calculating the conversion ratios of the CO and the CVP; step S6 of measuring the CO and the CVP during a treatment stage in a general ward; step S7 of converting the second measurement data D21 and D22 measured by the general ward device 200 into values of the CO and the CVP that are estimated based on the assumption that the CO and the CVP are being measured by the ICU device 100; and step S8 of displaying the estimated value and the measurement value in a graph. Hereinafter, the management method will be described in detail.

First, the ICU device 100 measures the CO and the CVP of the patient P1 during the treatment stage in the ICU (step S1). The measurement is performed at predetermined time intervals, for example. The acquisition unit 311 acquires the first measurement data D11 and D12 of the CO and the CVP from the ICU device 100, and stores the first measurement data D11 and D12 in the storage unit 320.

Then, the ICU device 100 and the general ward device 200 measure the CO and the CVP of the patient P1 at the same time on a day when the patient P1 leaves the ICU and moves to the general ward (step S2). At this time, the measurements are performed at the same time by attaching both measurement devices of the ICU device 100 and the general ward device 200 to the body of the patient P1. However, after the measurement by the ICU device 100 is performed, the measurement by the general ward device 200 may be performed within the same general period as the measurement by the ICU device 100. The acquisition unit 311 acquires the measurement data D11, D12, D21, and D22 from the ICU device 100 and the general ward device 200.

Then, the conversion unit 312 calculates the conversion ratio of the CO shown in the above Expression 2 using the first measurement data D11 and the second measurement data D21 of the CO measured in step S2 (step S3). In addition, the conversion unit 312 calculates the conversion ratio of the CVP shown in the above Expression 4 using the first measurement data D12 and the second measurement data D22 of the CVP measured in step S2.

Then, the CPU 310 determines whether or not the value of the conversion ratio of each of the CO and the CVP calculated in step S3 is within the reference range (step S4). The reference range is not limited to a particular range so long as the accuracy of conversion into the estimated value is within a range of a predetermined level or higher.

When it is determined that the conversion ratio of each of the CO and the CVP is outside the reference range (step S4; No), the CPU 310 determines whether or not the number of repetitions of step S4 is equal to or greater than a predetermined number of times (step S41). When it is determined that the number of repetitions of step S4 has not reached the predetermined number of times (step S41; No), the CPU 310 notifies the user, such as the doctor P2 or the nurse, that the conversion ratio is out of the reference range, and instructs the user to adjust the installation state of the general ward device 200 (step S42).

After adjusting the installation state of the general ward device 200, the CPU 310 performs steps S2 to S4 again. The CPU 310 repeats steps S2 to S4, step S41, and step S42 until the conversion ratio of each of the CO and the CVP falls within the reference range or the number of repetitions of step S4 reaches a predetermined number of times or more. Therefore, it is possible to prevent the accuracy of conversion to the estimated value from being lowered due to the inappropriate installation state of the general ward device 200. In addition, by adjusting the installation state of the general ward device 200 with the ICU device 100 (pulmonary artery catheter 110) having excellent measurement accuracy as a reference, the general ward device 200 can measure the CO and the CVP more accurately.

When it is determined that the conversion ratio of each of the CO and the CVP is within the reference range (step S4; Yes) or the number of repetitions of step S4 is a predetermined number of times or more (step S41; Yes), the CPU 310 instructs the user, such as the doctor P2 or the nurse, to remove the ICU device 100 (step S5). The user such as the doctor P2 or the nurse removes the ICU device 100 according to the instruction and moves the patient P1 to the general ward.

Then, the general ward device 200 measures the CO and the CVP of the patient P1 during the treatment stage in the general ward (step S6). The measurement is performed at predetermined time intervals, for example. The acquisition unit 311 acquires the second measurement data D21 and D22 of the CO and the CVP from the general ward device 200.

Then, as shown in the above Expression 1, the conversion unit 312 multiplies the second measurement data D21 of the CO measured in step S6 by the conversion ratio of the CO calculated in step S3 to convert the second measurement data D21 of the CO measured by the general ward device 200 into a value of the CO that is estimated based on the assumption that the CO is being measured by the ICU device 100 (step S7). In addition, as shown in the above Expression 3, the conversion unit 312 multiplies the second measurement data D22 of the CVP measured in step S6 by the conversion ratio of the CVP calculated in step S3 to convert the second measurement data D22 of the CVP measured by the general ward device 200 into a value of the CVP that is estimated based on the assumption that the CVP is being measured by the ICU device 100.

Since the ICU device 100 and the general ward device 200 have different measurement principles, measurement methods, and the like, even if the same circulatory parameter is measured at the same time, a difference between the measurement values may occur. The management system 10 can correct such a difference by performing conversion into the estimated value as described above. As a result, the measurement data D11, D12, D21, and D22 measured by the ICU device 100 and the general ward device 200 can be treated in the same manner as when these are measured by the same measurement device. Therefore, according to the management system 10, it is possible to track a series of changes in circulatory parameters during the treatment stage in the ICU and the treatment stage in the general ward. Therefore, a medical staff such as a doctor can easily determine a treatment policy. In addition, since the general ward device 200 is corrected in accordance with the measurement value of the ICU device 100 having high measurement accuracy, patients can be managed using the data with high accuracy.

Then, as shown in FIG. 4, the display control unit 313 displays points corresponding to the CO measurement value and the CVP measurement value of the ICU device 100 and points corresponding to the CO estimated value and the CVP estimated value of the general ward device 200 in a graph in which the vertical axis indicates the CO and the horizontal axis indicates the CVP (step S8). Then, the CPU 310 transmits the graph to the operation terminal S or the like of the user, such as the doctor P2. The CPU 310 may perform classification according to the degree of measurement accuracy of the general ward device 200 based on the conversion ratios of the CO and the CVP and transmit the result to the operation terminal S of the doctor P2 or the like. Therefore, the user such as the doctor P2 can easily track a series of changes in circulatory parameters during the treatment stage in the ICU and the treatment stage in the general ward. In addition, at this time, if it is determined in step S41 that the number of repetitions of step S4 is equal to or greater than the predetermined number of times, the CPU 310 notifies the doctor P2 or the like that the accuracy of conversion into the estimated value may be low.

Steps S6 to S8 illustrated in FIG. 5 may be repeatedly performed during the treatment stage in the general ward.

As described above, the management system 10 according to the first embodiment is a heart failure patient management system. The management system 10 includes the acquisition unit 311 and the conversion unit 312. The acquisition unit 311 acquires the first measurement data D11 and D12 of the first circulatory parameter related to the symptoms of heart failure from the ICU device 100 used during the heart failure treatment stage, and the second measurement data D21 and D22 of the second circulatory parameter related to the symptoms of heart failure from the general ward device 200 used during the heart failure treatment stage. Based on the correspondence between the first measurement data D11 and D12 and the second measurement data D21 and D22 measured at the same time among the first measurement data D11 and D12 and the second measurement data D21 and D22, the conversion unit 312 converts the second measurement data D21 and D22 of the second circulatory parameter measured by the general ward device 200 into values of the first circulatory parameter that are estimated based on the assumption that the CO and the CVP are being measured by the ICU device 100.

According to the management system 10 described above, it is possible to monitor a series of changes in the circulatory parameters measured by the ICU device 100 and the general ward device 200.

In addition, when the first circulatory parameter and the second circulatory parameter are expected to be the same, the conversion unit 312 calculates Conversion Expressions Y1 and Y2 for converting the second measurement data D21 and D22 measured by the general ward device 200 into values of the CO and the CVP that are estimated based on the assumption that the CO and the CVP are being measured by the ICU device 100, using the first measurement data D11 and D12 and the second measurement data D21 and D22 measured at the same time. As described above, the management system 10 can correct a difference caused by the difference between the measurement methods of the ICU device 100 and the general ward device 200 by performing conversion into the estimated values using Conversion Expressions Y1 and Y2.

In addition, the first circulatory parameter and the second circulatory parameter include a circulatory parameter indicating the cardiac output or the cardiac function and/or a circulatory parameter indicating the preload or afterload of the heart. Therefore, according to the management system 10, it is possible to monitor a series of changes in the cardiac output or the cardiac function and/or the preload or afterload of the heart.

In addition, the first measurement data D11 and D12 include the measurement data D11 of the circulatory parameter indicating the cardiac output or the cardiac function and the measurement data D12 of the circulatory parameter indicating the preload or afterload of the heart. The second measurement data D21 and D22 include the measurement data D21 of the circulatory parameter indicating the cardiac output or the cardiac function and the measurement data D22 of the circulatory parameter indicating the preload or afterload of the heart. Therefore, according to the management system 10, it is possible to monitor a series of changes in the circulatory parameter indicating the cardiac output or the cardiac function and the circulatory parameter indicating the preload or afterload of the heart. Therefore, the user such as the doctor P2 can diagnose heart failure more easily compared with a case where either one parameter of the circulatory parameter indicating the cardiac output or the cardiac function or the circulatory parameter indicating the preload or afterload of the heart is measured.

In addition, the management system 10 includes the display control unit 313 that displays, in a graph, values of the first circulatory parameter that are estimated based on the assumption that the CO and the CVP of the second circulatory parameter as measured by the general ward device 200 are being measured by the ICU device 100, and the values of the first circulatory parameter that are measured by the ICU device 100. Therefore, the user such as the doctor P2 can monitor a series of changes in circulatory parameters with reference to the graph.

In addition, the first measurement data D11 and D12 and the second measurement data D21 and D22 include the measurement data D11 and D21 of the circulatory parameter indicating the cardiac output or the cardiac function and the measurement data D12 and D22 of the circulatory parameter indicating the preload or afterload of the heart. The display control unit 313 displays measurement values and estimated values in a graph in which the vertical axis indicates a circulatory parameter indicating the cardiac output or the cardiac function and the horizontal axis indicates a circulatory parameter indicating the preload or afterload of the heart. Therefore, the user such as the doctor P2 can monitor a series of changes in the circulatory parameter indicating the cardiac output or the cardiac function and the circulatory parameter indicating the preload or afterload of the heart with reference to one graph.

In addition, the ICU device 100 is an invasive measurement device, and the general ward device 200 is a non-invasive measurement device. In this manner, the management system 10 can correct the difference between the invasive measurement device and the non-invasive measurement device.

In addition, the ICU device 100 includes the pulmonary artery catheter 110. Therefore, according to the management system 10, it is possible to correct the difference between the measurement data D11 and D12 of the circulatory parameter measured by the pulmonary artery catheter 110 and the measurement data of the circulatory parameter measured by another measurement device due to a measurement method difference or the like.

In addition, the management method according to the first embodiment is a management method for a patient with heart failure. In the management method, the first measurement data D11 and D12 of the first circulatory parameter related to the symptoms of heart failure is acquired from the ICU device 100 used during the heart failure treatment stage, and the second measurement data D21 and D22 of the second circulatory parameter related to the symptoms of heart failure is acquired from the general ward device 200 used during the heart failure treatment stage. Based on the correspondence between the first measurement data D11 and D12 and the second measurement data D21 and D22 measured at the same time, the second measurement data D21 and D22 of the second circulatory parameter measured by the general ward device 200 is converted into values of the first circulatory parameter that are estimated based on the assumption that the CO and the CVP are being measured by the ICU device 100.

In addition, the management program according to the first embodiment is a management program for a patient with heart failure. The management program executes: a procedure for acquiring the first measurement data D11 and D12 of the first circulatory parameter related to the symptoms of heart failure from the ICU device 100 used during the heart failure treatment stage and the second measurement data D21 and D22 of the second circulatory parameter related to the symptoms of heart failure from the general ward device 200 used during the heart failure treatment stage; and a procedure for converting the second measurement data D21 and D22 of the second circulatory parameter measured by the general ward device 200 into values of the first circulatory parameter that are estimated based on the assumption that the CO and the CVP are being measured by the ICU device 100 based on the correspondence between the first measurement data D11 and D12 and the second measurement data D21 and D22 measured at the same time.

According to the management method and the management program described above, it is possible to monitor a series of changes in the circulatory parameters measured by the ICU device 100 and the general ward device 200.

### Second Embodiment

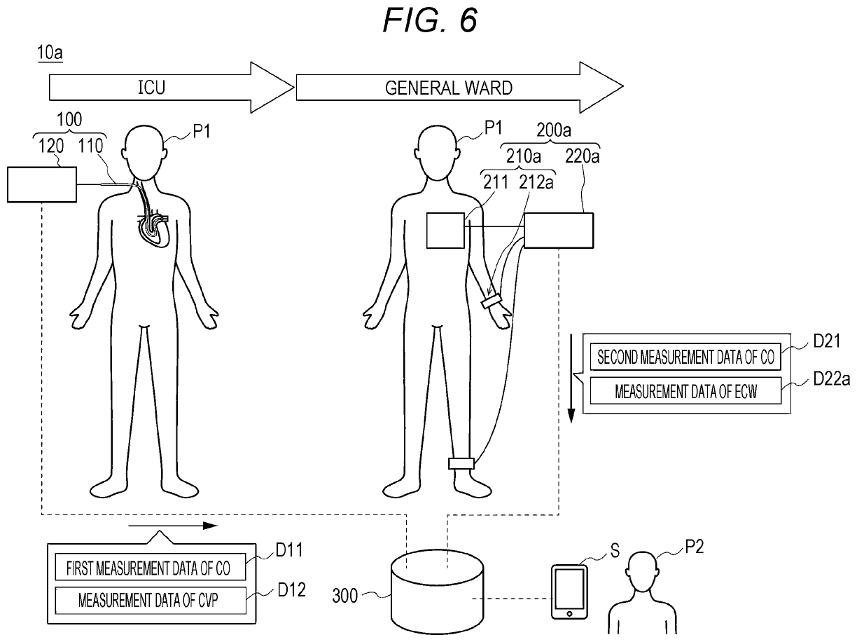

FIGS. 6 to 8 are diagrams describing a management system 10a according to a second embodiment.

The management system 10a and the management method according to the second embodiment are different from the management system 10 and the management method according to the first embodiment in that a general ward device 200a measures the ECW as a circulatory parameter indicating the preload or afterload of the heart. Hereinafter, the management system 10a and the management method according to the second embodiment will be described. In addition, the same components as in the management system 10 according to the first embodiment are denoted by the same reference numerals except for the conversion unit 312 and the display control unit 313, and the description thereof will be omitted.

(General Ward Device)

As illustrated in FIG. 6, in the present embodiment, the general ward device 200a includes a measurement unit 210a configured to measure a circulatory parameter indicating the cardiac output or the cardiac function and a circulatory parameter indicating the preload or afterload of the heart and a control unit 220a that is electrically connected to the measurement unit 210a and controls the operation of the measurement unit 210a. The details will be described below.

The measurement unit 210a includes a CO measurement unit 211 configured to measure the CO as a circulatory parameter indicating the cardiac output or the cardiac function and an ECW measurement unit 212a configured to measure the ECW as a circulatory parameter indicating the preload or afterload of the heart.

The ECW measurement unit 212a is not limited to a particular device, and is for example, a known non-invasive ECW measurement device, such as a bioimpedance measurement device. As described above, in the present embodiment, the general ward device 200 measures a circulatory parameter (CO) indicating the cardiac output or the cardiac function, which is the same as that for the ICU device 100, and measures a circulatory parameter (ECW) indicating the preload or afterload of the heart, which is different from that for the ICU device 100.

The control unit 220a is configured as a known microcomputer including a CPU, a ROM, and a RAM.

The control unit 220a is electrically connected to the CO measurement unit 211 and the ECW measurement unit 212a, and controls the measurement operations of the CO measurement unit 211 and the ECW measurement unit 212a. The control unit 220a causes the CO measurement unit 211 and the ECW measurement unit 212a to measure the CO and the ECW while the patient P1 is being treated in a general ward. The control unit 220a transmits, to the server 300, second measurement data D21 of the CO and measurement data of the ECW measured by the measurement unit 210a.

(Conversion Unit)

Based on the correspondence between the first measurement data D11 and the second measurement data D21 of the CO measured at the same time, the conversion unit 312 converts the second measurement data D21 of the CO measured by the general ward device 200a into a value of the CO that is estimated based on the assumption that the CO is being measured by the ICU device 100 (hereinafter, referred to as “CO-CO conversion”). Based on the correspondence between the measurement data D12 of the CVP and the measurement data D22a of the ECW measured at the same time, the conversion unit 312 converts the measurement data D12 of the CVP measured by the ICU device 100 into a value of the ECW that is estimated based on the assumption that the CVP is being measured by the general ward device 200 (hereinafter, referred to as “CVP-ECW conversion”). In addition, in the CO-CO conversion, the ICU device 100 corresponds to the first measurement device, and the general ward device 200a corresponds to the second measurement device. In the CVP-ECW conversion, the ICU device 100 corresponds to the second measurement device, and the general ward device 200a corresponds to the first measurement device. In addition, in the CVP-ECW conversion, the CVP measured by the ICU device 100 corresponds to the second circulatory parameter, and the ECW measured by the general ward device 200a corresponds to the first circulatory parameter.

Since the CO-CO conversion method is the same as the conversion method in the first embodiment, the description thereof will be omitted (refer to the above Expressions 1 and 2). Hereinafter, the CVP-ECW conversion method will be described.

During heart failure, as a result of a compensatory mechanism with respect to a decline in cardiac function, the amount of body fluid increases, and congestion occurs in the pulmonary vein, central vein, and the like (that is, ECW increases). As a result, the CVP increases. Thus, during heart failure, there is a correlation between the ECW and the CVP. According to research done by the present inventors, such a correlation between the CVP and the ECW can be expressed by predetermined Relational Expression F1. In addition, in the present embodiment, a case where Relational Expression F1 is a linear function as shown in the following Expression 5 will be described as an example. However, Relational Expression F1 is not limited to a linear function so long as this represents the correlation between the ECW and the CVP. Relational Expression F1 is stored in the storage unit 320.

[Expression 5]

Relational expression F1:ECW=a₁×CVP+b (Formula 5)

a₁: Predetermined coefficient
b: Predetermined intercept

The conversion unit 312 calculates parameter conversion values by converting the measurement data D12 of the CVP of the ICU device 100 into the ECW using the above Expression 5. Therefore, it is possible to normalize the circulatory parameters to the ECW.

Using the parameter conversion value derived from the measurement data D12 of the CVP and the measurement data D22a of the ECW, both the CVP and the ECW being measured at the same time, the conversion unit 312 calculates Conversion Expression Y3 for converting the parameter conversion value into a value of the ECW that is estimated based on the assumption that the parameter conversion value is being derived from CVP that is measured by the general ward device 200a. In the present embodiment, as shown in the following Expression 6, a conversion method will be described by taking a case, in which Conversion Expression Y3 is a linear function (proportional expression) having an intercept of 0, as an example. In addition, Conversion Expression Y3 may be any expression as long as the parameter conversion value can be converted into a value of the ECW, and is not limited to the linear function.

[Expression 6]

Conversion expression Y3: Estimated value of ECW=Conversion ratio of ECW×Parameter conversion value (Formula 6)

As shown in the following Expression 7, the conversion unit 312 calculates the ratio between the parameter conversion value derived from the measurement data D12 of the CVP and the measurement data D22a of the ECW, both the CVP and the ECW being measured at the same time, e.g., time T0, as the conversion ratio of the ECW.

$$\begin{matrix} [ Expression 7 ] & \\ Conversion ratio of ECW = \frac{\begin{matrix}Measurement data D 22 a of \\ ECW measured at time T 0\end{matrix}}{\begin{matrix}Parameter conversion value derived from \\ measurement data D 12 of CVP measured at \\ time T 0\end{matrix}} & ( Formula 7 )\end{matrix}$$

As shown in the above Expression 6, the conversion unit 312 sets a value, which is obtained by multiplying the parameter conversion value derived from the measurement data D12 of the CVP by the conversion ratio of the ECW, as a value of the ECW that is estimated based on the assumption that the CVP is being measured by the general ward device 200a.

In addition, although the conversion unit 312 converts the measurement data D12 of the CVP into an estimated value of the ECW in the present embodiment, the conversion unit 312 may convert the measurement data D22a of the ECW into an estimated value of the CVP. In addition, the conversion unit 312 may perform both conversion of the measurement data D12 of the CVP into the estimated value of the ECW and conversion of the measurement data D22a of the ECW into the estimated value of the CVP.

As the conversion ratios of the CO and the ECW deviate from 1, the measurement value of the general ward device 200a deviates from the measurement value of the ICU device 100 having higher measurement accuracy. That is, the conversion ratios of the CO and the ECW indicate the measurement accuracy of the general ward device 200a relative to the measurement accuracy of the ICU device 100. The CPU 310 may perform classification according to the degree of measurement accuracy of the general ward device 200a based on the conversion ratios of the CO and the ECW and transmit the result to the operation terminal S of the doctor P2 or the like.

(Display Control Unit)

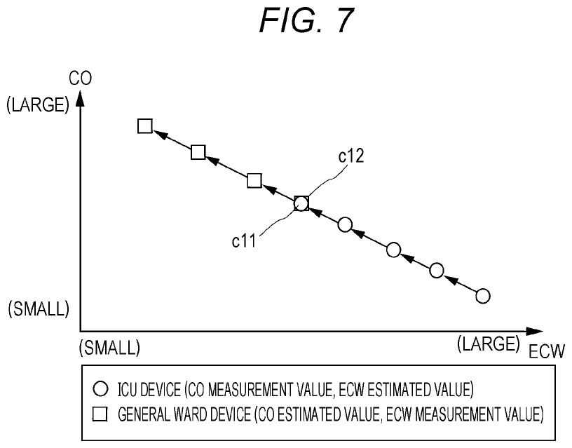

As shown in FIG. 7, the display control unit 313 displays points (indicated by white circles in FIG. 7) corresponding to the CO measurement values and the ECW estimated values of the ICU device 100 and points (indicated by white quadrangles in FIG. 7) corresponding to the CO estimated values and the ECW measurement values of the general ward device 200a in a graph, in which the vertical axis (corresponding to “first axis”) indicates the CO and the horizontal axis (corresponding to “second axis”) indicates the ECW. In addition, the horizontal axis may indicate the CO, and the vertical axis may indicate the ECW.

In addition, the graph shown in FIG. 7 is merely an example. For example, in FIG. 7, a point c11 of the ICU device 100 and a point c12 of the general ward device 200a, which represent measurements carried out at the same time, are both shown. However, just one of the points c11 and c12 may be displayed.

In addition, in FIG. 7, the points of the ICU device 100 and the points of the general ward device 200a are displayed with different shapes (circle and quadrangle). However, the points of the ICU device 100 and the general ward device 200a may be displayed with the same shape. In addition, in FIG. 7, the changes of the points with time are indicated by arrows. However, the arrows indicating the change with time may be omitted in the graph. In addition, although FIG. 7 shows a case where the ECW decreases with time and the CO increases with time, the changes in the ECW and the CO are not limited to the case shown in the diagram.

(Management Method)

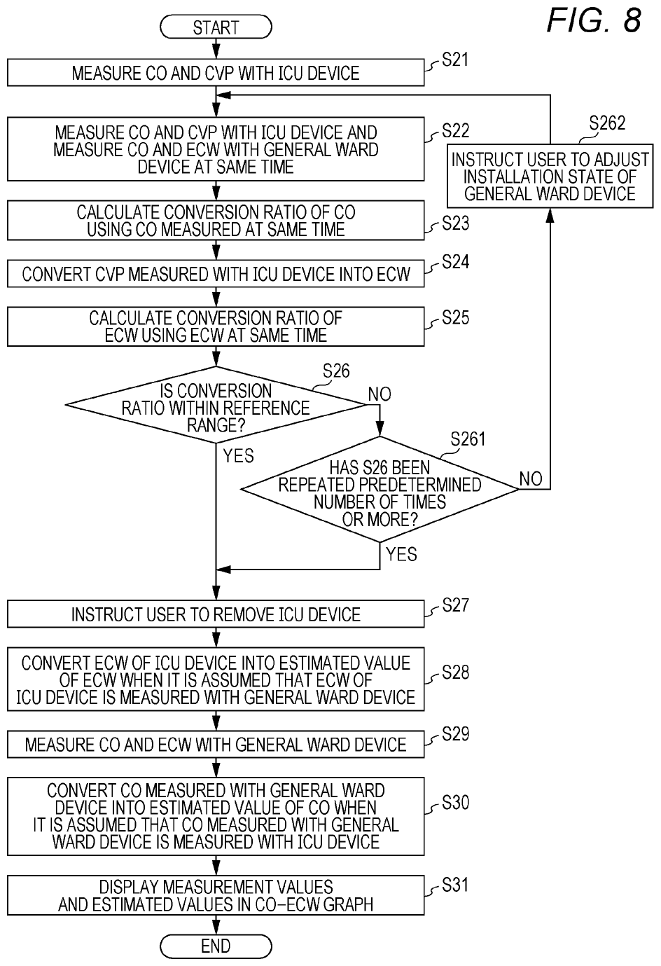

The management method according to the present embodiment will be briefly described with reference to FIG. 8. The management method includes: step S21 of measuring the CO and the CVP during a treatment stage in the ICU; steps S22 to S27 of calculating the conversion ratios of the CO and the CVP; step S28 of converting the measurement data D12 of the CVP measured by the ICU device 100 into a value of the ECW that is estimated based on the assumption that the CVP is being measured by the general ward device 200a; step S29 of measuring the CO and the ECW during a treatment stage in a general ward; step S30 of converting the second measurement data D21 of the CO measured by the general ward device 200a into a value of the CO that is estimated based on the assumption that the CO is being measured by the ICU device 100; and step S31 of displaying the measurement value and the estimated value in a graph. Hereinafter, the management method will be described in detail.

First, the ICU device 100 measures the CO and the CVP of the patient P1 during the treatment stage in the ICU (step S21). The measurement is performed at predetermined time intervals, for example. The acquisition unit 311 acquires the first measurement data D11 of the CO and the measurement data D12 of the CVP from the ICU device 100.

Then, on a day when the patient P1 leaves the ICU and moves to the general ward, the ICU device 100 measures the CO and the CVP of the patient P1, and the general ward device 200a measures the CO and the ECW of the patient P1 at the same time as the measurement by the ICU device 100 (step S22). The measurements are performed at the same time by attaching both the ICU device 100 and the general ward device 200a to the body of the patient P1. However, alternatively, after the measurement by the ICU device 100 is performed, the measurement by the general ward device 200a may be performed within the same general time period as the measurement by the ICU device 100. The acquisition unit 311 acquires the measurement data D11, D12, D21, and D22a from the ICU device 100 and the general ward device 200a.

Then, the conversion unit 312 calculates the conversion ratio of the CO in the above Expression 2 using the first measurement data D11 and the second measurement data D21 of the CO measured in step S22 (step S23).

Then, the conversion unit 312 calculates parameter conversion values by converting the measurement data D12 of the CVP measured in steps S21 and S22 into the ECW using the above Expression 5 (Relational Expression F1) (step S24).

Then, the conversion unit 312 calculates the conversion ratio of the ECW in the above Expression 7 using the parameter conversion value derived from the measurement data D12 of the CVP measured at the same time as the general ward device 200a and the measurement data D22a of the ECW measured at the same time among the parameter conversion values calculated in step S24 (step S25).

Then, the CPU 310 determines whether or not the value of the conversion ratio of each of the CO and the ECW calculated in steps S23 and S25 is within the reference range (step S26). The reference range is not limited to a particular range so long as the accuracy of conversion into the estimated value is within a range of a predetermined level or higher.

When it is determined that the conversion ratio of each of the CO and the ECW is outside the reference range (step S26; No), the CPU 310 checks whether or not the number of repetitions of step S26 is equal to or greater than a predetermined number of times (step S261). When it is determined that the number of repetitions of step S26 has not reached the predetermined number of times (step S261; No), the CPU 310 instructs the user, such as the doctor P2 or the nurse, to adjust the installation state of the general ward device 200a (step S262). After adjusting the installation state of the general ward device 200a, the CPU 310 performs steps S22 to S26 again. The CPU 310 repeats steps S22 to S26, step S261, and step S262 until the conversion ratio falls within the reference range or the number of repetitions of step S26 reaches a predetermined number of times or more.

When it is determined that the conversion ratio is within the reference range (step S26; Yes) or when the number of repetitions of step S26 is a predetermined number of times or more (step S261; Yes), the CPU 310 instructs the user, such as the doctor P2 or the nurse, to remove the ICU device 100 (step S27). The doctor P2 or the nurse removes the ICU device 100 according to the instruction and moves the patient P1 to the general ward.

Then, as shown in the above Expression 6, the conversion unit 312 multiplies the parameter conversion value calculated in step S24 by the conversion ratio of the ECW to convert the measurement data D12 of the CVP of the ICU device 100 into a value of the ECW that is estimated based on the assumption that the CVP is being measured by the general ward device 200a (step S28).

Then, the general ward device 200a measures the CO and the ECW of the patient P1 during the treatment stage in the general ward (step S29). The measurement is performed at predetermined time intervals, for example. The acquisition unit 311 acquires the second measurement data D21 of the CO and the measurement data D22a of the ECW from the general ward device 200a.

Then, as shown in the above Expression 1, the conversion unit 312 multiplies the second measurement data D21 of the CO measured in step S29 by the conversion ratio of the CO calculated in step S23 to convert the second measurement data D21 of the CO measured by the general ward device 200a into a value of the CO that is estimated based on the assumption that the CO is being measured by the ICU device 100 (step S30).

Then, as shown in FIG. 7, the display control unit 313 displays points corresponding to the CO measurement value and the ECW measurement value of the ICU device 100 and points corresponding to the CO estimated value and the ECW measurement value of the general ward device 200a in a graph in which the vertical axis indicates the CO and the horizontal axis indicates the ECW (step S31). Then, the CPU 310 transmits the graph to the operation terminal S of the user, such as the doctor P2. The CPU 310 may perform classification according to the degree of measurement accuracy of the general ward device 200a based on the conversion ratios of the CO and the ECW and transmit the result to the operation terminal S of the doctor P2 or the like. In addition, at this time, if it is determined in step S261 that the number of repetitions of step S26 is equal to or greater than the predetermined number of times, the CPU 310 notifies the doctor P2 or the like that the accuracy of conversion into the estimated value may be low.

Steps S29 to S31 may be repeatedly performed during the treatment stage in the general ward. In addition, before step S23, steps S24 and S25 may be performed, or step S23 and steps S24 and S25 may be performed simultaneously in parallel. In addition, when the determination result in step S26 is No, step S262 may be performed next without performing step S261. In addition, the timing at which step S28 is performed is not limited to a particular timing so long as the timing is after Yes is determined in step S26 or S261 and is before step S31.

As described above, in the management system 10a according to the second embodiment, when the first circulatory parameter (ECW) and the second circulatory parameter (CVP) are different, the conversion unit 312 calculates a parameter conversion value by converting the measurement data D12 of the CVP into the ECW value using Relational Expression F1 indicating the correlation between the CVP and the ECW. Using the parameter conversion values measured at the same time among the parameter conversion values and the ECW measurement data D22a of the general ward device 200a measured at the same time, the conversion unit 312 calculates Conversion Expression Y3 for converting the parameter conversion value into a value of the ECW that is estimated based on the assumption that the parameter conversion value is being derived from CVP that is measured by the general ward device 200a.

According to the management system 10a described above, even if circulatory parameters to be measured by the ICU device 100 and the general ward device 200a are different, it is possible to monitor a series of changes in the circulatory parameters in the ICU treatment stage and the general ward treatment stage by normalizing the parameters.

In addition, in the second embodiment, the case has been described in which the combination of circulatory parameters indicating the preload or afterload of the heart measured by the ICU device 100 and the general ward device 200a is the CVP and the ECW. However, the combination of circulatory parameters indicating the preload or afterload of the heart measured by the ICU device 100 and the general ward device 200a is not limited to a particular combination so long as there is a correlation between parameters measured by the ICU device 100 and the general ward device 200a. For example, the combination of circulatory parameters indicating the preload or afterload of the heart measured by the ICU device 100 and the general ward device 200a may be the PCWP and the ECW, may be the PAP and the ECW, or may be the CVP and the ICW.

### Third Embodiment

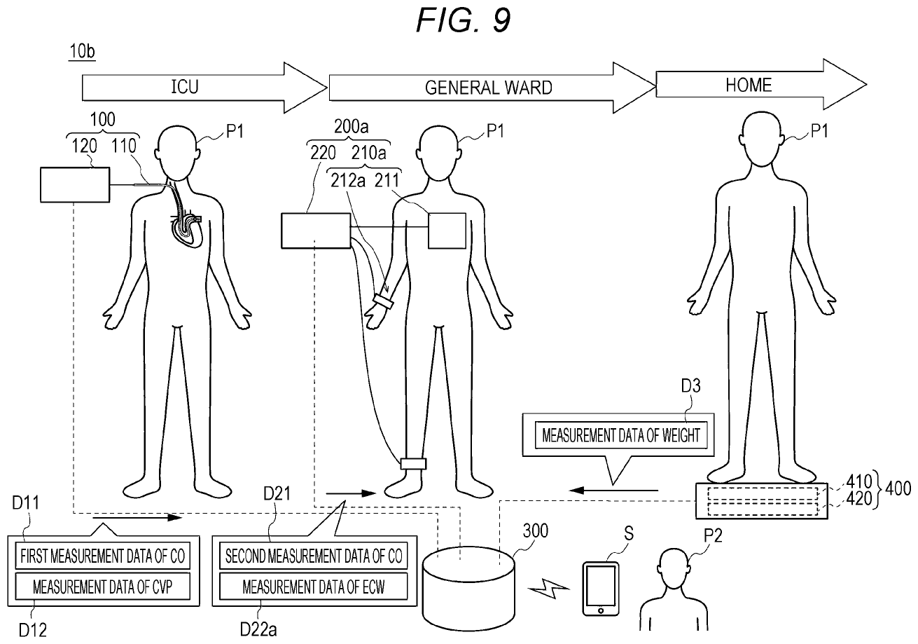

FIGS. 9 to 11B are diagrams describing a management system 10b according to a third embodiment.

The management system 10b and the management method according to the third embodiment are different from the management system 10 and the management method according to the first embodiment and the management system 10a and the management method according to the second embodiment in that the patient P1 is managed during a treatment stage (third treatment stage) at home after leaving the medical institution from the treatment stage in the ICU. Hereinafter, the management system 10b and the management method according to the third embodiment will be described. In addition, the same components as in the management system 10 according to the first embodiment and the management system 10a according to the second embodiment are denoted by the same reference numerals except for the conversion unit 312 and the display control unit 313, and the description thereof will be omitted.

The management system 10b according to the third embodiment will be briefly described with reference to FIG. 9. The management system 10b according to the third embodiment includes an ICU device 100, a general ward device 200a, a home device 400 used during a treatment stage at home, and a server 300 that is connected to the ICU device 100, the general ward device 200a, the home device 400, and the operation terminal S of the doctor P2 through a network (shown by the broken line in the diagram) to perform transmission and reception of data between the ICU device 100, the general ward device 200a, the home device 400, and the operation terminal S of the doctor P2.

In this manner, the management system 10b also manages the treatment stage at home. The patient is discharged from the hospital when the symptoms of heart failure subside and the ADL that can return to daily life is achieved. However, due to various factors such as excessive salt or water intake at home and forgetting to take the drug, acute exacerbation is likely to occur again. For this reason, the patient regularly visits the outpatient clinic and receives appropriate treatment. At this time, it is useful to manage the condition of the patient by the management system 10b.

(Home Device)

In the present embodiment, the home device 400 includes a measurement unit 410 that measures a circulatory parameter indicating the preload or afterload of the heart and a control unit 420 that is electrically connected to the measurement unit 410 and controls the operation of the measurement unit 410. The details will be described below.

In the present embodiment, the measurement unit 410 is configured by a known scale configured to measure the weight of the patient P1 as a circulatory parameter indicating the preload or afterload of the heart. In addition, the circulatory parameter indicating the preload or afterload of the heart measured by the measurement unit 410 is not limited to a particular circulatory parameter so long as there is a correlation with a circulatory parameter indicating the preload or afterload of the heart measured by the general ward device 200a.

The control unit 420 includes a known microcomputer including a CPU, a ROM, a RAM, and the like. The control unit 420 transmits the measurement data D3 of the weight measured by the measurement unit 410 to the server 300.

(Conversion Unit)

Based on the correspondence between the first measurement data D11 and the second measurement data D21 of the CO measured at the same time, the conversion unit 312 converts the second measurement data D21 of the CO measured by the general ward device 200a into a value of the CO that is estimated based on the assumption that the CO is being measured by the ICU device 100 (CO-CO conversion). Based on the correspondence between the measurement data D12 of the CVP and the measurement data D22a of the ECW measured at the same time, the conversion unit 312 converts the measurement data D12 of the CVP measured by the ICU device 100 into a value of the ECW that is estimated based on the assumption that the CVP is being measured by the general ward device 200 (“CVP-ECW conversion”). Based on the correspondence between the measurement data D22a of the ECW and the measurement data D3 of the weight measured at the same time, the conversion unit 312 converts the measurement data D3 of the weight measured by the home device 400 into a value of the ECW that is estimated based on the assumption that the weight is being measured by the general ward device 200 (hereinafter, referred to as “weight-ECW conversion”).

In addition, since the CO-CO conversion method and the CVP-ECW conversion method are the same as the methods described in the first and second embodiments (refer to the above Expressions 1 to 7 and the like), the description thereof will be omitted. Hereinafter, the weight-ECW conversion method will be described.

During heart failure, as a result of a compensatory mechanism with respect to a decline in cardiac function that occurs to compensate for a decrease in cardiac output, the body fluid increases, and blood accumulates in the pulmonary vein, central vein, and the like (that is, ECW increases). As a result, the weight of the patient increases. Thus, there is a correlation between the ECW and the weight. It is known that such a correlation between the ECW and the weight can be expressed by Relational Expression F2 as shown in the following Expression 8. Relational Expression F2 is stored in the storage unit 320.

[Expression 8]

Relational expression F2=ECW=a₂×Weight

a₂: Predetermined coefficient (for example, in case of normal male, a₂=6/25) (Formula 8)

The conversion unit 312 calculates parameter conversion values by converting the weight measurement data D3 of the home device 400 into a value of the ECW using the above Expression 8. Therefore, it is possible to normalize the circulatory parameters.

Using the parameter conversion value derived from the measurement data D3 of the weight measured at the same time and the measurement data D22a of the ECW measured at the same time, the conversion unit 312 calculates Conversion Expression Y4 for converting the parameter conversion value into a value of the ECW that is estimated based on the assumption that the parameter conversion value is being derived from weight that is measured by the general ward device 200a. In the present embodiment, as shown in the following Expression 9, a conversion method will be described by taking a case, in which Conversion Expression Y4 is a linear function (proportional expression) having an intercept of 0, as an example. In addition, Conversion Expression Y4 may be any expression as long as the parameter conversion value can be converted into a value of the ECW, and is not limited to a linear function.

[Expression 9]

Conversion expression Y4: Estimated value of ECW=Conversion ratio of ECW×Parameter conversion value (Formula 9)

As shown in the following Expression 10, the conversion unit 312 calculates the ratio between the parameter conversion value derived from the measurement data D3 of the weight and the measurement data D22a of the ECW, both the weight and the ECW being measured at the same time, e.g., time T0, as the conversion ratio of the ECW.

$$\begin{matrix} [ Expression 10 ] & \\ Conversion ratio of ECW = \frac{\begin{matrix}Measurement data D 22 a of ECW \\ measured at time T 0\end{matrix}}{\begin{matrix}Parameter conversion value derived from \\ measurement data D 3 of weight measured \\ at time T 0\end{matrix}} & ( Formula 10 )\end{matrix}$$

As shown in the above Expression 9, the conversion unit 312 sets a value, which is obtained by multiplying the parameter conversion value derived from the measurement data D3 of the weight by the conversion ratio of the ECW, as a value of the ECW that is estimated based on the assumption that the weight is being measured by the general ward device 200a.

In addition, although the conversion unit 312 converts the measurement data D3 of the weight into the estimated value of the ECW in the present embodiment, the conversion unit 312 may convert the measurement data D22a of the ECW into the estimated value of the weight, or may perform both conversion of the measurement data D3 of the weight into the estimated value of the ECW and conversion of the measurement data D22a of the ECW into the estimated value of the weight.

As the conversion ratio of the ECW deviates from 1, the measurement value of the home device 400 deviates from the measurement value of the general ward device 200a having higher measurement accuracy. That is, the conversion ratio of the ECW indicates the measurement accuracy of the home device 400 relative to the measurement accuracy of the general ward device 200a. The CPU 310 may perform classification according to the degree of measurement accuracy of the home device 400 based on the conversion ratio of the ECW and transmit the result to the operation terminal S of the doctor P2 or the like.

(Display Control Unit)

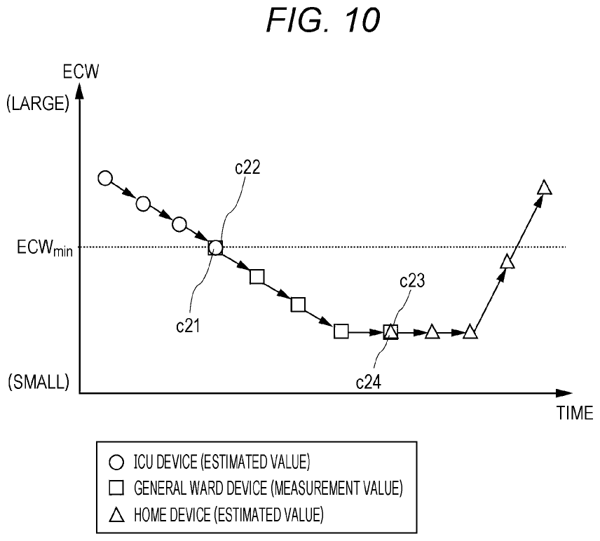

As shown in FIG. 10, the display control unit 313 displays points (indicated by white circles in FIG. 10) corresponding to the ECW estimated values of the ICU device 100, points (indicated by white quadrangles in FIG. 10) corresponding to the ECW measurement values of the general ward device 200a, and points (indicated by white triangles in FIG. 10) corresponding to the ECW estimated values of the home device 400 in a graph in which the vertical axis indicates the ECW and the horizontal axis indicates time (or the number of times of measurement).

In addition, the graph shown in FIG. 10 is merely an example. For example, in FIG. 10, a point c21 corresponding to the ECW of the ICU device 100 and a point c22 corresponding to the ECW of the general ward device 200a, which represent measurements carried out at the same time, are both shown. However, just one of the points c21 and c22 may be displayed. In addition, in FIG. 10, a point c23 corresponding to the ECW of the general ward device 200a and a point c24 corresponding to the ECW of the home device 400, which represent measurements carried out at the same time, are both shown. However, just one of the points c23 and c24 may be displayed.

In addition, in FIG. 10, the points of the ICU device 100, the points of the general ward device 200a, and the points of the home device 400 are displayed with different shapes (circle, quadrangle, and triangle). However, the points of the ICU device 100, the points of the general ward device 200a, and the points of the home device 400 may be displayed with the same shape. In addition, in FIG. 10, changes with time are indicated by arrows. However, the arrows indicating the change with time may be omitted in the graph. In addition, although FIG. 10 shows a case where the ECW decreases with time during the treatment stage from the ICU to the general ward and the ECW increases with time in the latter half of the treatment stage at home, the change in the ECW is not limited to the case shown in the diagram.

(Management Method)

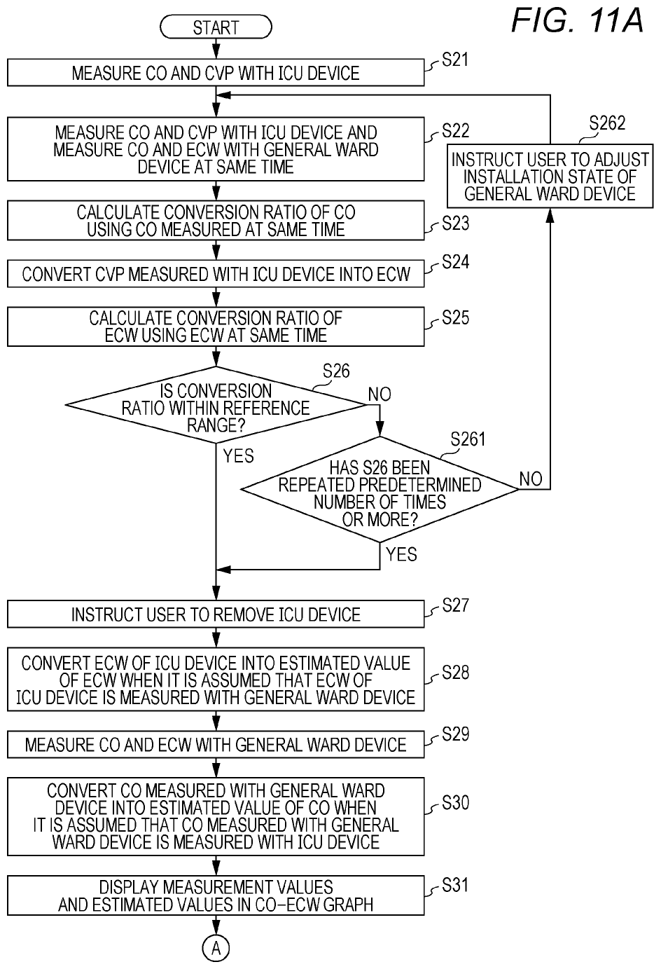

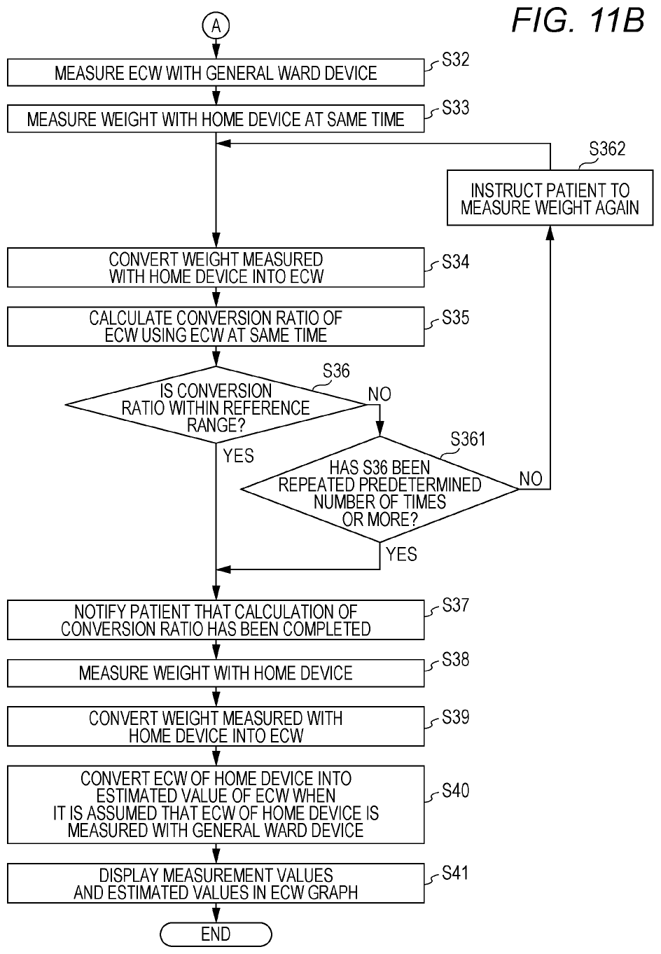

Next, the management method according to the third embodiment will be described with reference to FIGS. 11A and 11B. In addition, since processing of steps S21 to S31 illustrated in FIG. 11A is the same as that in the second embodiment, the description thereof will be omitted.

As illustrated in FIG. 11B, the general ward device 200a measures the ECW of the patient P1 on the day of discharge (step S32). The acquisition unit 311 acquires the measurement data D22a of the ECW from the general ward device 200a.

Then, the home device 400 measures the weight of the patient P1 at the same time as step S32 (for example, the same day as the day on which step S32 is performed) (step S33). The acquisition unit 311 acquires the measurement data D3 of the weight from the home device 400.

Then, the conversion unit 312 calculates parameter conversion values by converting the measurement data D3 of the weight measured in step S33 into the value of the ECW using Relational Expression F2 (above Expression 8) (step S34).

Then, the conversion unit 312 calculates the conversion ratio of the ECW in Expression 10 using the parameter conversion value calculated in step S34 and the measurement data D22a of the ECW measured in step S32 (step S35).

Then, the CPU 310 determines whether or not the value of the conversion ratio of the ECW calculated in step S35 is within the reference range (step S36).

When it is determined that the conversion ratio is outside the reference range (step S36; No), the CPU 310 checks whether or not the number of repetitions of step S36 is equal to or greater than a predetermined number of times (step S361). When it is determined that the number of repetitions of step S36 has not reached the predetermined number of times (step S361; No), the CPU 310 instructs the patient P1 to measure the weight again, and the home device 400 measures the weight of the patient P1 again (S362). Then, the CPU 310 performs steps S34 to S36 again. The CPU 310 repeats steps S34 to S36, step S361, and step S362 until the conversion ratio falls within the reference range or the number of repetitions of step S36 reaches a predetermined number of times or more.

When it is determined that the conversion ratio is within the reference range (step S36; Yes) or when the number of repetitions of step S36 is a predetermined number of times or more (step S361; Yes), the CPU 310 notifies the patient P1 that the calculation of the ECW conversion ratio has been completed (step S37).

Then, the home device 400 measures the weight of the patient P1 during the treatment stage at home (step S38). The measurement is performed at predetermined time intervals, for example. The acquisition unit 311 acquires the measurement data D3 of the weight from the home device 400.

Then, the conversion unit 312 calculates parameter conversion values by converting the measurement data D3 of the weight measured in step S38 into the value of the ECW using Relational Expression F2 (above Expression 8) (step S39).

Then, as shown in the above Expression 9, the conversion unit 312 multiplies the parameter conversion value calculated in step S39 by the conversion ratio of the ECW calculated in step S35 to convert the weight measurement data D3 of the home device 400 into a value of the ECW that is estimated based on the assumption that the weight is being measured by the general ward device 200a (step S40). At this time, when the calculated estimated value of the ECW exceeds a minimum value ECW min (refer to FIG. 10) of the estimated value of the ECW during the treatment stage in the ICU, the CPU 310 may warn the patient P1 to immediately visit the medical institution. In addition, the CPU 310 may determine the risk of re-hospitalization during the treatment stage at home by comparing data at the time of hospitalization (the treatment stage in the ICU and the treatment stage in the general ward) with the data during the treatment stage at home. Then, the risk of re-hospitalization may be presented to the patient P1, the doctor P2, or the like.

Then, as shown in FIG. 10, the display control unit 313 displays points corresponding to the ECW estimated value of the ICU device 100, points corresponding to the measurement value of the second measurement data D22a of the ECW of the general ward device 200a, and points corresponding to the ECW estimated value of the home device 400 in a graph in which the vertical axis indicates the ECW and the horizontal axis indicates time or the number of times of measurement (step S41). Then, the CPU 310 transmits the graph to the operation terminal S of the doctor P2. The CPU 310 may perform classification according to the degree of measurement accuracy of the home device 400 based on the conversion ratio of the ECW and transmit the result to the operation terminal S of the doctor P2 or the like. In addition, at this time, if it is determined in step S36 that the conversion ratio is outside the reference range, the CPU 310 notifies the doctor P2 or the like that the accuracy of conversion into the estimated value may be low.

Steps S38 to S41 may be repeatedly performed during the treatment stage at home.

As described above, in the management system 10b according to the third embodiment, the acquisition unit 311 acquires the measurement data D3 of the circulatory parameter related to the symptoms of heart failure from the home device 400 used during the treatment stage at home, and the conversion unit 312 converts measurement data measured by at least one of the general ward device 200a and the home device 400 into a value that is estimated based on the assumption that the measurements are being made by the other measurement device, based on the correspondence between the ECW measurement data D22a and the weight measurement data D3 acquired at the same time.

According to the management system 10b, as described in the first and second embodiments, it is possible to monitor a series of changes in circulatory parameters during the treatment stage in the ICU and the treatment stage in the general ward. In addition, according to the above-described management system 10b, it is possible to monitor a series of changes in circulatory parameters during the treatment stage in the general ward and the treatment stage at home. Therefore, more accurate patient management can be realized in the ICU, general ward, and home.

While the present invention has been described above through the embodiments, the scope of the present disclosure is not limited to the respective configurations described above, and can be appropriately modified based on the description of the claims.

For example, the means and method for performing various kinds of processing in the management system may be realized by a dedicated hardware circuit or a programmed computer.

In addition, for example, in the embodiments described above, the server 300 of the management systems 10, 10a, and 10b has been described as being realized as one device, but the scope of the present disclosure is not limited thereto. For example, the server 300 may be configured to include a plurality of servers, or may be virtually configured by a large number of servers installed at remote locations as cloud servers.

In addition, for example, in the embodiments described above, the form in which the server 300 functions as an acquisition unit, a conversion unit, and a display control unit has been described. However, for example, any one of the general ward devices 200 and 200a, the operation terminal S of the user such as the doctor P2, and the operation terminal of the patient may function as an acquisition unit, a conversion unit, and a display control unit.

In addition, for example, in the third embodiment described above, the form in which the management system 10b manages the treatment stage in ICU to the treatment stage at home (three treatment stages) has been described. However, the management system 10b may manage only the treatment stage in the general ward and the treatment stage at home (two treatment stages).

In addition, for example, the first treatment stage may be a treatment stage in a general ward, and the second treatment stage may be a treatment stage in a rehabilitation room. In this case, a person who teaches rehabilitation can more appropriately set the exercise load during rehabilitation using a series of data provided by the management system.

In addition, for example, in the embodiments described above, the measurement data of the “non-invasive measurement device” is converted into a value that is estimated based on the assumption that the measurement is carried out by the “invasive measurement device”. However, the measurement data of the “invasive measurement device” may be converted into a value that is estimated based on the assumption that the measurement is carried out by the “non-invasive measurement device”. In addition, the measurement data of the “non-invasive measurement device that does not require much time to measure” may be converted into a value that is estimated based on the assumption that the measurement is carried out by the “non-invasive measurement device that requires more time to measure and has higher accuracy”. In addition, the measurement data of the “invasive but less invasive measurement device” may be converted into a value that is estimated based on the assumption that the measurement is carried out by the “invasive but more invasive and more accurate measurement device”.

What is claimed is:

1. A management system for managing a patient, comprising:
an acquisition unit configured to acquire at a plurality of timings, first measurement data of a first circulatory parameter related to symptoms of heart failure from a first measurement device, and second measurement data of a second circulatory parameter related to symptoms of heart failure from a second measurement device; and
a conversion unit configured to convert the second measurement data of the second circulatory parameter measured by the second measurement device into an estimated value of the first circulatory parameter, based on correspondence between the first measurement data and the second measurement data that are measured at substantially the same time.

2. The management system according to claim 1,
wherein the first circulatory parameter and the second circulatory parameter are the same circulatory parameter, and
the conversion unit is configured to calculate a conversion expression for converting the second measurement data into the estimated value using the first measurement data and the second measurement data measured at substantially the same time.

3. The management system according to claim 1,
wherein the first circulatory parameter and the second circulatory parameter are different circulatory parameters, and
the conversion unit is configured to:
calculate a parameter conversion value by converting the second measurement data into a value of the first circulatory parameter using a relational expression indicating a correlation between the first circulatory parameter and the second circulatory parameter, and
calculate a conversion expression for converting the parameter conversion value into the estimated value using the first measurement data and the parameter conversion value converted from the second measurement data measured at substantially the same time as the first measurement data.

4. The management system according to claim 1, wherein the first circulatory parameter and the second circulatory parameter include a circulatory parameter indicating a cardiac output or a cardiac function and a circulatory parameter indicating a preload or afterload of the heart.

5. The management system according to claim 4,
wherein the first measurement data includes measurement data of the circulatory parameter indicating the cardiac output or the cardiac function and measurement data of the circulatory parameter indicating the preload or afterload of the heart, and
the second measurement data includes measurement data of the circulatory parameter indicating the cardiac output or the cardiac function and measurement data of the circulatory parameter indicating the preload or afterload of the heart.

6. The management system according to claim 1, further comprising:
a display control unit configured to generate a graph that displays the estimated value of the first circulatory parameter and a measurement value of the first circulatory parameter.

7. The management system according to claim 6,
wherein the first measurement data and the second measurement data include measurement data of the circulatory parameter indicating the cardiac output or the cardiac function and measurement data of the circulatory parameter indicating the preload or afterload of the heart, and
the graph displays the circulatory parameter indicating the cardiac output or the cardiac function on a first axis and the circulatory parameter indicating the preload or afterload of the heart on a second axis.

8. The management system according to claim 1, wherein the first measurement device is an invasive measurement device, and the second measurement device is a non-invasive measurement device.

9. The management system according to claim 8, wherein the first measurement device is a pulmonary artery catheter.

10. A management method for a patient, comprising:
acquiring at a plurality of timings first measurement data of a first circulatory parameter related to symptoms of heart failure from a first measurement device and second measurement data of a second circulatory parameter related to symptoms of heart failure from a second measurement device; and
converting the second measurement data of the second circulatory parameter measured by the second measurement device into an estimated value of the first circulatory parameter, based on correspondence between the first measurement data and the second measurement data measured at substantially the same time.

11. The management method according to claim 10, wherein the first measurement device is an invasive measurement device, and the second measurement device is a non-invasive measurement device.

12. The management method according to claim 11, wherein the first measurement device is a pulmonary artery catheter.

13. A non-transitory computer readable medium in which a management program for monitoring a patient is stored, wherein the management program is executable on a processor to carry out:
a procedure for acquiring at a plurality of timings first measurement data of a first circulatory parameter related to symptoms of heart failure from a first measurement device and second measurement data of a second circulatory parameter related to symptoms of heart failure from a second measurement device; and
a procedure for converting the second measurement data of the second circulatory parameter measured by the second measurement device into an estimated value of the first circulatory parameter, based on correspondence between the first measurement data and the second measurement data measured at substantially the same time.

14. The non-transitory computer readable medium according to claim 13, wherein the first measurement device is an invasive measurement device, and the second measurement device is a non-invasive measurement device.

15. The non-transitory computer readable medium according to claim 14, wherein the first measurement device is a pulmonary artery catheter.

16. A patient monitoring server comprising:
a communication interface connected to a communication network;
a storage unit configured to store a plurality of first measurement data of a first circulatory parameter related to symptoms of heart failure of the patient received over the communication network, and a plurality of second measurement data of a second circulatory parameter related to symptoms of heart failure of the patient received over the communication network; and
a processor configured to convert the second measurement data of the second circulatory parameter into estimated values of the first circulatory parameter, based on correspondence between the first measurement data and the second measurement data that were measured at substantially the same time, and generate a display showing at least one measured first circulatory parameter and at least one estimated value of the first circulatory parameter.

17. The patient monitoring server according to claim 16, wherein the generated display is transmitted to a computing device of a person monitoring the patient.

18. The patient monitoring server according to claim 17,
wherein the first measurement data and the second measurement data include measurement data of the circulatory parameter indicating the cardiac output or the cardiac function and measurement data of the circulatory parameter indicating the preload or afterload of the heart.

19. The patient monitoring server according to claim 18,
the graph displays the circulatory parameter indicating the cardiac output or the cardiac function on a first axis and the circulatory parameter indicating the preload or afterload of the heart on a second axis.

20. The patient monitoring server according to claim 16, wherein the first measurement data has higher accuracy than the second measurement data.
